(19) United States
(12) Patent Application Publication
(10) Pub. No.: US 2019/0183398 A1
(43) Pub. Date: Jun. 20, 2019

(54) BIOFLUID SENSING DEVICES WITH TEMPERATURE REGULATION

(71) Applicant: Eccrine Systems, Inc., Cincinnati, OH (US)

(72) Inventors: Jason Heikenfeld, Cincinnati, OH (US); Mikel Larson, Cincinnati, OH (US); Nathan Weinle, Fairfield, OH (US)

(73) Assignee: Eccrine Systems, Inc., Cincinnati, OH (US)

(21) Appl. No.: 16/326,317

(22) PCT Filed: Aug. 21, 2017
(86) PCT No.: PCT/US17/47808

§ 371 Date: Feb. 18, 2019

Related U.S. Application Data
(60) Provisional application No. 62/377,090, filed on Aug. 19, 2016.

(51) Int. Cl. A61B 5/145; A61B 5/01; A61B 5/00

(52) U.S. Cl. CPC A61B 5/14546; A61B 5/01; A61B 5/1451; A61B 2010/0067; A61B 5/4277; A61B 5/4272; A61B 5/14517

(57) ABSTRACT

The disclosed invention includes a biofluid sensing device capable of passively or actively regulating an operating temperature of one or more sensors. The device includes at least one biofluid sensor in a thermally isolated environment and at least one temperature sensor to measure sensor environment temperature. Some embodiments include at least one thermal adjustor to regulate the sensor temperature by actively regulating the sensor environment temperature in response to a signal from the temperature sensor. The invention also includes a method of regulating temperature for a biofluid sensing device having a sensor for measuring an analyte in the biofluid. The method includes measuring a biofluid sensor temperature, regulating the sensor temperature to within a selected range of the measured sensor temperature, and maintaining sensor temperature within the selected range of the measured temperature throughout device operation. In some embodiments, the measured temperature is a calibration temperature.

### CROSS-REFERENCE TO RELATED APPLICATIONS

The present application claims priority to PCT/US17/47808, filed Aug. 21, 2017, and U.S. Provisional Application No. 62/377,090, filed Aug. 19, 2016, the disclosures of which are hereby incorporated by reference herein in their entirety.

### BACKGROUND OF THE INVENTION

Non-invasive biosensing technologies have enormous potential for numerous applications including athletics, neonatology, pharmacological monitoring, and personal health. Biofluids, such as sweat, provide access to many of the same biomarkers, chemicals, and solutes that are found in blood, which can enable the diagnosis of ailments, health status, toxins, physical exertion, and other physiological attributes in the absence of any physical symptoms. Sweat has many of the same analytes and analyte concentrations as found in blood and interstitial fluid. Interstitial fluid has even more analytes nearer to blood concentrations than sweat does, especially for larger-sized and more hydrophilic analytes (such as proteins).

However, one challenge in using sweat to diagnosis physiological conditions is that the electrical signals from biofluid sensors often have a strong dependence on temperature. For example, ion selective electrode (ISE) sensors, have electrical outputs that are governed by the Nernst equation:

$${E}_{red} = {E}_{red}^{θ} + \frac{RT}{zF} ln \frac{{a}_{Ox}}{{a}_{Red}} ,$$

where T represents temperature. For ISEs, increasing temperatures will increase the total potential change between two given concentrations. This increased potential change can improve sensor performance by increasing the total bandwidth of the signal. While increasing temperatures can improve sensor performance, temperature variations, even at higher average temperatures, can compromise sensor performance. In particular, variations in sensor temperatures can have an adverse effect on the accuracy of an ISE sensor's response to a target analyte. Similarly, the output of enzymatic sensors, and other sensor modalities is based upon temperature-dependent, kinetic reaction rates. In particular, temperature and temperature variation can have significant effects on electrochemical aptamer-based (EAB) sensors, which are discussed in U.S. Pat. Nos. 7,803,542 and 8,003,374, and U.S. Provisional Application No. 62/523,835, filed Jun. 23, 2017, each of which is hereby incorporated by reference herein in its entirety. For EAB sensors, temperature directly affects the kinetic equilibrium between aptamers and the target analyte concentration in a biofluid sample, and temperature influences structural variations within the aptamer. Accordingly, small changes in temperature will impact the accuracy of single point calibrations by shifting the binding affinity of the aptamer to its target analyte. Likewise, large temperature changes can impact the secondary and tertiary structures of the aptamer, altering the aptamer's pre-capture presentation, and post-capture conformational response to its target analyte.

One solution to the challenge of temperature variation is to add a temperature sensor to a biofluid sensing device, and use the sensor output to correct for temperature-induced errors in the biofluid sensor output signal. For example, integration of a temperature sensor in a sweat sensing device is disclosed in PCT/US13/35092, which is incorporated herein by reference in its entirety. However, for many biofluid sensing applications, simply adding sensors to correct for temperature errors may prove inferior to passively or actively regulating the temperature of the biofluid sensor(s). Furthermore, a temperature sensor may not accurately measure a biofluid sensor temperature without the sensor being thermally isolated from the device wearer's body or the external environment. Accordingly, it is desirable to have biofluid sensing devices which passively or actively regulate the operating temperature of one or more biofluid sensors; as well as simple, yet robust, methods for regulating the operating temperature of one or more sensors in a biofluid sensing device.

### SUMMARY OF THE INVENTION

The disclosed invention includes a biofluid sensing device capable of passively or actively regulating an operating temperature of one or more sensors. The device includes at least one biofluid sensor in a thermally isolated environment and at least one temperature sensor to measure sensor environment temperature. Some embodiments include at least one thermal component to regulate the sensor temperature by actively adjusting the sensor environment temperature in response to a signal from the temperature sensor. The invention also includes a method of regulating temperature for a biofluid sensing device having at least one sensor for measuring an analyte in the biofluid. The method includes measuring the biofluid sensor environment, and regulating the biofluid sensor environment temperature to within a selected range of the measured sensor temperature. The method further includes maintaining sensor temperature within the selected range of the measured temperature throughout device operation. In some embodiments, the measured temperature is a calibration temperature.

### BRIEF DESCRIPTION OF THE DRAWINGS

The present disclosure will be further appreciated in light of the following detailed description and drawings in which:

FIG. 1 is a cross-sectional, diagrammatic view of at least a portion of a first exemplary embodiment of a wearable biosensing device configured to regulate temperature for at least one biofluid sensor.

FIG. 2 is a cross-sectional, diagrammatic view of at least a portion of a second exemplary embodiment of a wearable biosensing device configured to regulate temperature for at least one biofluid sensor.

FIG. 3 is a cross-sectional, diagrammatic view of at least a portion of a third exemplary embodiment of a wearable biosensing device configured to regulate temperature for at least one biofluid sensor.

FIG. 4 is a cross-sectional, diagrammatic view of at least a portion of a fourth exemplary embodiment of a wearable biosensing device configured to regulate temperature for at least one biofluid sensor.

### DEFINITIONS

Before continuing with a detailed description of the exemplary embodiments, a variety of definitions should be made, these definitions gaining further appreciation and scope in the detailed description of the embodiments.

As used herein, “interstitial fluid” is a solution that bathes and surrounds tissue cells. The interstitial fluid is found in the spaces between cells. Embodiments of the disclosed invention measure analytes from interstitial fluid found in the skin and, particularly, interstitial fluid found in the dermis. In some cases, where interstitial fluid is emerging from sweat ducts, the interstitial fluid contains some sweat as well, or alternately, sweat may contain some interstitial fluid. As used herein, “mainly interstitial fluid” means fluid that contains by volume less than 50% sweat (i.e., is primarily interstitial fluid). As used herein, “mainly sweat” means fluid that contains by volume 50% or greater of sweat (i.e., may contain some interstitial fluid, but has an equal or greater amount of sweat than interstitial fluid). The percentages of each fluid can be quantified by several methods, such as measuring analyte dilutions in sweat (e.g., some analytes are dilute in sweat but not in interstitial fluid), or by measuring and comparing sample generation rates and their respective contributions to the total fluid volume (e.g., compare sample generation rates with or without application of reverse iontophoresis; or compare sample generation rates with or without natural or chemically-induced sweat stimulation).

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

“Biofluid” means any human biofluid, including, without limitation, sweat, interstitial fluid, blood, plasma, serum, tears, and saliva.

As used herein, “chronological assurance” means the sampling rate or sampling interval that assures measurement(s) of analytes in a biofluid at the rate where measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30-minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, “biofluid sampling rate”, “sweat sampling rate”, or simply “sampling rate” means the effective rate at which a new biofluid sample reaches a sensor that measures a property of the fluid or its solutes. Sampling rate is the rate at which new biofluid is refreshed at the one or more sensors, with the new biofluid displacing the old biofluid as the new fluid arrives. In one embodiment, the sampling rate can be estimated based on volume, flow-rate, and time calculations, although it is recognized that some biofluid or solute mixing can occur. Sampling rate directly determines, or is a contributing factor in determining, the chronological assurance. Sampling times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval”. Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluids, or other measurement contamination sources for the measurement(s). Sampling rate can also be in part determined from solute generation, transport, advective transport of fluid, diffusion transport of solutes, or other factors that will impact the rate at which new samples will reach a sensor and/or is altered by older sample or solutes or other contamination sources.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “microfluidic components” are channels or other geometries formed in or by polymers, textiles, paper, or other components known in the art to transport fluid in a deterministic manner.

As used herein, “sweat sample channel” means any component of the disclosed invention that is on or adjacent to a sweat sensing device sweat sample collector and that promotes transport of sweat or its solutes by wicking pressure, advective flow, diffusion, or other method of transport, from the collector, across device sensors and to a sweat sample pump. In some embodiments, the channel function may be performed by a suitably configured sweat collector. A channel may be part of the same component or material that serves other purposes (e.g., a sweat collector or a sweat sample pump), and in such cases, the portion of said component or material that, at least in part, fluidically connects the collector to the pump and conveys sweat to a sensor(s) and that is on or adjacent to the sensor(s), is also a channel as defined herein.

As used herein, the term “analyte-specific sensor” is a sensor specific to an analyte which performs specific chemical recognition of the analyte's presence or concentration (e.g., ion-selective electrodes, enzymatic sensors, electrochemical aptamer-based sensors, etc.). Sensors could also be optical, mechanical, or use other physical/chemical methods which are specific to a single analyte. Further, multiple sensors can each be specific to one of multiple analytes.

### DETAILED DESCRIPTION OF THE INVENTION

Turning now to the drawing figures, in which like numerals refer to like features throughout the views, several exemplary embodiments will be described of a wearable sensing device for measuring at least one analyte in a sweat or other biofluid sample. The sensing device measures samples at chronologically assured sampling rates or intervals. The sensing device embodiments described herein can take on many forms including patches, bands, straps, portions of clothing, wearables, or any suitable mechanism that reliably brings sampling and sensing technology into intimate proximity with a biofluid sample as it is transported to the skin surface. While some sensing device embodiments utilize adhesives to hold the device near the skin, devices could also be held by other mechanisms that hold the device secure against the skin, such as a strap or embedding in a helmet. Embodiments herein depict one or more of the sensors as simple individual elements. However, it should be understood that many sensors require two or more electrodes, reference electrodes, or additional supporting technology or features, which are not captured in the description herein, but which may be included in the device embodiments. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sensing devices with more sub-components needed for use of the device in various applications. Where these sub-components would be obvious (such as a battery), for purposes of brevity and of greater focus on inventive aspects, such components are not explicitly shown in the figures or described in the embodiments.

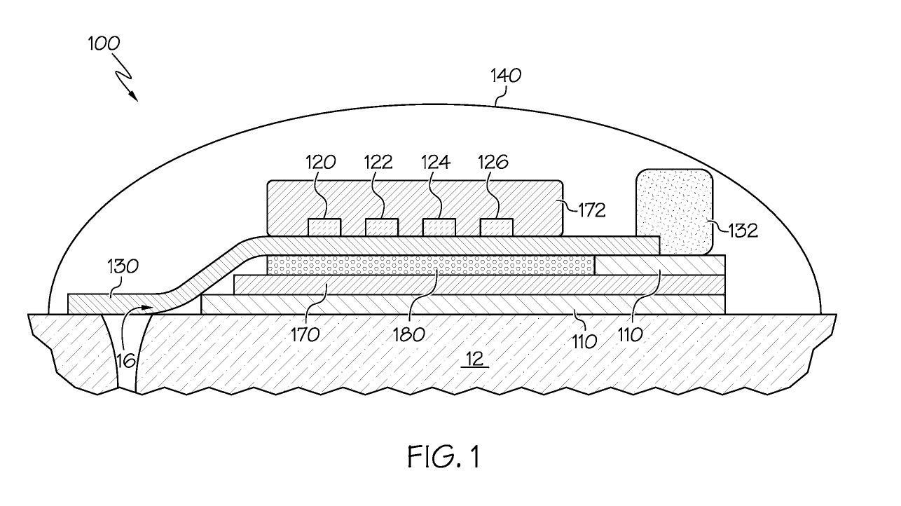

With reference to FIG. 1, in a first exemplary embodiment at least a portion of a biofluid sensing device 100 is placed on skin 12. The device 100 includes at least one biofluid sensor 120 and at least one temperature sensor 122. Sensor 120 could be specific to analytes such as, for example, Na⁺, K⁺ (ion-selective electrodes), glucose (enzymatic/amperometric sensor), corticosteroids, proteins, and/or viruses (electrochemical, aptamer-based sensor). Temperature sensor 122 could be, for example, a thermocouple or a thermistor, such as a 10 k resistor, which is capable of accurately measuring temperature within, for example, ±0.25° C. The device 100 may include additional biofluid sensors or temperature sensors 124, 126. Generally, biofluid sensors 120, 124 are placed in fluid communication with a microfluidic wick or channel 130, and temperature sensors 122, 126 are placed within the local environment of the biofluid sensors. Multiple temperature sensors, or higher resolution sensors, could be used in the disclosed embodiments to increase accuracy.

The device 100 further includes a polymer substrate 110, which could be made of PET or other suitable material. An adhesive (not shown) may extend between the substrate 110 and the skin 12 to attach the device to the wearer. A protective vapor barrier layer 140 extends about the exterior of the device. Outer layer 140 is shown only in the first embodiment (FIG. 1), however, this outer layer would typically also be present in the other embodiments described below. The microfluidic channel or wick 130 carries biofluid 16 from the surface of skin 12, to the sensors 120, 122, 124, 126, and onto a pump 132, by any suitable mechanism for transport, including wicking pressures (e.g., component 130 and pump 132 are a paper or textile), or that transports sweat via capillary pressure or via pressure produced by sweat glands (e.g., component 130 is a channel).

The device 100 also includes a passive thermal component comprising one or more layers of a low thermal conductivity material or insulator, as indicated at 170 and 172. The insulators 170, 172 may include any one or more of the following materials: cotton (0.04 W/m-K); an air-filled space, such as a container or silica aerogel (0.02 W/m-K); foam glass or glass wool (0.4 W/m-K); Styrofoam (0.3 W/m-K); a vacuum vessel (<0.01 W/m-K), or other materials. Additionally, the insulators 170, 172 may range in thickness from approximately 0.01 mm to 10 mm, but may be thicker than 10 mm in some embodiments. The insulators 170, 172 thermally isolate the sensors 120, 122, 124, 126 in an environment separate from atmospheric or ambient air. The thermal isolation decreases the temperature variability of the sensors 120, 122, 124, 126, enabling the sensors to operate at a consistent, controlled temperature. While the biofluid sensors 120, 124 and temperature sensors 122, 126 are depicted as being separated by intervening insulator material, the invention is not so limited, and some embodiments include configurations where biofluid sensors and temperature sensors are not separated by insulator material.

An active thermal component, consisting of a heater 180, is provided in thermal communication with the sensors 120, 122, 124, 126. The heater 180 may, for example, be a resistive heater, an infrared light emitting diode, or a thermoelectric module, and is placed in thermal communication with the sensors 120, 122, 124, 126, so that generated heat is transferred to the sensor environment. In at least some embodiments, the heater 180 regulates the temperature of the sensors to within a range of ±5° C. to ±0.1° C. of 40° C. (106° F.). In at least some embodiments, the heater 180 regulates the temperature of the sensors to one of the following temperatures: >37° C.; >39° C.; >41° C.; and >45° C.

The heater 180 enables the sensing device 100 to raise and maintain a sensor temperature above the device wearer's body temperature. Maintaining a high sensor temperature is advantageous for a number of reasons. Certain biofluid sensors, such as ISE sensors, tend to be more accurate at higher temperatures. Other sensor types, such as EAB sensors, provide a stronger response (greater signal change) to analyte concentration changes when operating at higher temperatures. Further, maintaining an elevated sensor temperature can reduce temperature variability, and hence sensor output variability, because maintaining a higher sensor temperature will be relatively easier than maintaining a lower sensor temperature for many device applications. This would apply to situations in which the wearer's body temperature or the external temperature increases during device use, e.g., the wearer begins to exercise after device application, the wearer develops a fever, or the device is applied when the external temperature is at a low point, etc. Therefore, if sensor temperature were lowered rather than raised, temperature-induced variability would likely increase.

In this embodiment, an insulating layer 170 extends between the heater 180 and the skin 12, while a second insulating layer 172 substantially surrounds the sensors 120, 122, 124, 126, thereby thermally isolating the sensors and heater 180 from the external environment. With the sensors located within or between the insulating layers 170, 172, changes in conditions outside of the insulating layers (e.g., changes in skin or body temperature, air temperature, etc.) will not immediately affect the sensors, which will be held at a nearly constant temperature by the low thermal conductivity material. If the wearer is using the device in an environment with high external temperatures, the heater 180 may be used to increase the regulated temperature to a temperature that is appropriate for the application.

With further reference to FIG. 1, the biofluid sensors 120, 124 may be calibrated for operation at a consistent, regulated temperature, or within a selected temperature range, i.e., within ±5° C. to ±0.1° C. of 40° C. (106° F.). The biofluid sensors 120, 124 are calibrated by exposing the sensors to a calibration solution having known analyte concentrations while the device is maintaining the sensor temperature at the regulated temperature. After a sensor is calibrated as described, the objective for regulating sensor temperature becomes maintaining consistency with the calibration temperature, meaning that the sensor environment should be maintained at least within a temperature range of the calibration temperature, and variations ought to be minimized.

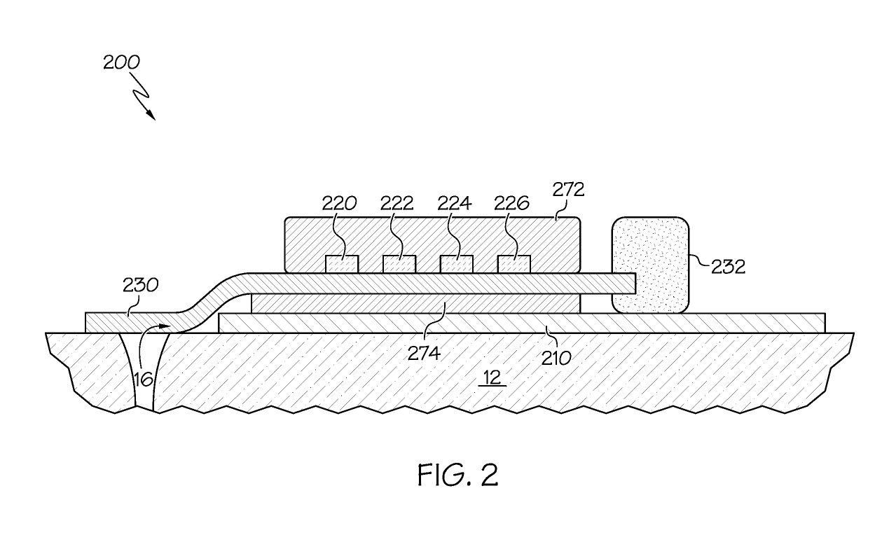

In an alternative embodiment, shown in FIG. 2, a modified sensing device 200 is depicted in which the heater 180 is omitted. In this embodiment, the device 200 includes a thermal component consisting of one or more layers of a thermal coupling material 274 for transferring heat from the skin 12 to the at least one sensor 220, 222, 224, 226. Thermal coupling material 274 is located between the skin 12 and the sensors 220, 222, 224, 226, and conveys body heat directly to the sensors through wicking component 230. In this configuration, the temperature of the biofluid sensor(s) 220, 224 is regulated by the wearer's body temperature, which likely changes less than the atmospheric or ambient temperature. As in the previous embodiment, a low thermal conductivity material 272 substantially surrounds the sensors 220, 222, 224, 226 to thermally isolate the sensors from atmospheric and ambient temperature variations. In some versions of this embodiment, the temperature sensor(s) 222, 226 can optionally be removed, as the biofluid sensor temperature is regulated directly from the wearer's body temperature.

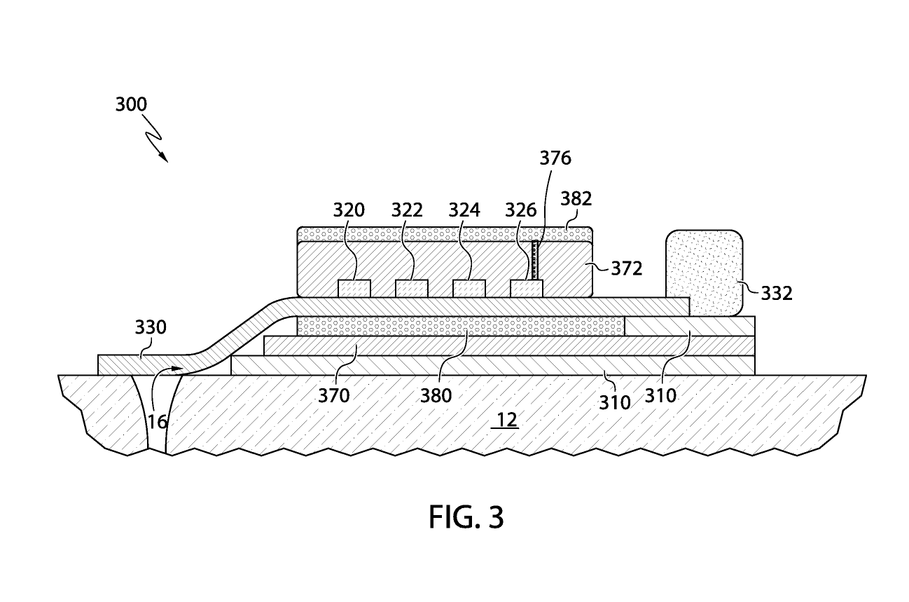

With reference to FIG. 3, in another alternative embodiment, a sensing device 300 is shown positioned on a section of skin 12 for detecting and measuring at least one analyte in a biofluid 16. In this embodiment, the device 300 is similar to the embodiment of FIG. 1, but includes an additional thermal component, consisting of a thermoelectric cooler 382, for decreasing the regulated temperature. Thermoelectric cooler 382 is operable by a controller (not shown) to decrease the temperature of the temperature regulated components, such as the sensors 320, 322, 324, 326, or a microfluidic or wicking component 330. In some versions of this embodiment, the low thermal conductivity layer 372 which surrounds the sensors includes a plurality of thermally conducting components 376 (one is shown) that extend between the thermoelectric cooler 382 and the sensors 326, so that each sensor is in thermal communication with the cooler 382. Generally, the thermal conductors 376 may be a metal, epoxy, or other material having a thermal conductivity greater than 1 W/m-K, and optimally has a thickness less than 1 mm. Some embodiments may have a plurality of thermal conductors (not shown), configured to facilitate thermal communication between the sensors and the heater 380.

With the addition of a thermoelectric cooler 382, the sensors 320, 322, 324, 326 can be regulated to remain below the body temperature (i.e., below around 99° F. or 30° C.), by transferring a lower temperature from thermoelectric cooler 382 through the thermally conducting material 376. The heater 380 and cooler 382 can be operated in conjunction with each other, or separately controlled in response to a signal from the temperature sensor 322 to maintain a consistent, regulated temperature regardless of increases or decreases in body temperature or ambient air temperatures.

With further reference to FIG. 3, the heater 380 and the thermoelectric cooler 382 are actively controlled using closed-loop feedback from the at least one temperature sensor 322. For applications requiring relatively less precision (e.g., ±5° C. or ±1° C.), a proportional controller alone could regulate the temperature at the various sensors. For applications requiring relatively greater precision (±0.2° C. or ±0.1° C.), a controller with proportional-integral-derivative (PID) feedback may be used. In either case, the controls are realized, and gains tuned, by firmware running on embedded electronics in the device (not shown). The embedded electronics are capable of taking measurements from the one or more temperature sensors 322, and supplying a variable power input to the heater 380 or thermoelectric cooler 382. The variable power may be in the form of pulse width modulation (PWM) or a true analog power variation, i.e., supplying a lower voltage, or lower current to the regulating components, to control heating or cooling output.

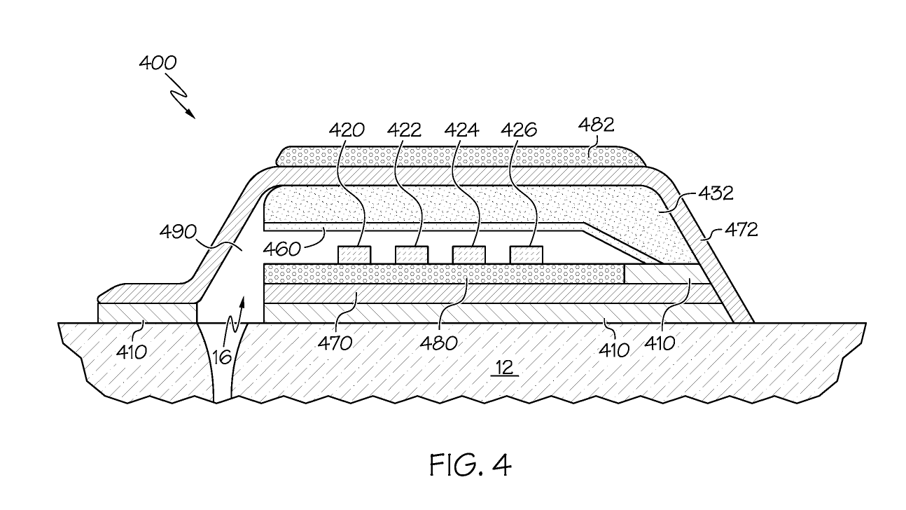

With reference to FIG. 4, an alternative biofluid sensing device 400 with temperature regulation is depicted. In this embodiment, the device 400 includes a sweat-impermeable substrate 410, having an opening to a concentrator channel 490 for collecting biofluid samples 16. The device 400 also includes at least one biofluid sensor 420, at least one temperature sensor 422, a concentrator membrane 460, and a pump 432. As in the previous embodiments, the device 400 can also include additional biofluid sensors and temperature sensors, as indicated generally at 424, 426. The concentrator membrane 460 may be a dialysis membrane, an osmosis membrane permeable to ions and impermeable to small molecules and proteins, or another type of membrane that is at least permeable to water and impermeable to the target analyte. An optional pre-sensor membrane (not shown), also made from similar material types as used for the concentrator membrane, may be provided to filter unwanted solutes, such as molecules larger than the target analyte, from the biofluid sample before it reaches sensors 420, 422, 424, 426. The pump 432 includes material to facilitate wicking or osmotic flow of the biofluid sample, which may be, for example, a hydrogel, textile, salt, polyelectrolyte solution, or desiccant, such as MgSO₄. In some embodiments, the pump 432 will have a significantly greater volume than the concentrator channel 490 to facilitate pH and salinity buffering of the biofluid sample. The device may be attached on skin 12 via an adhesive (not shown). As biofluid 16 enters the device 400 and flows into the concentrator channel 490, water, and in some cases untargeted solutes, are drawn through the membrane 460, and into the pump 432. Such sample manipulation leaves the target analyte molecules in the concentrator channel 490, and effectively concentrates the biofluid sample with respect to the target analyte.

To regulate the temperature of the sensors 420, 422, 424, 426, the device 400 further includes one or more thermal components. As shown in FIG. 4, these thermal components can include a heater 480, similar to the heater described for previous embodiments, and/or a thermoelectric cooler 482, also as described for previous embodiments. One or more low thermal conductivity layers 470, 472 are also provided. In some versions of this embodiment, as described for previous embodiments, the low thermal conductivity layer 472 includes a plurality of thermally conducting components (not shown) that extend between the thermoelectric cooler 482 and the concentrator channel 490 or sensors 420, 422, 424, 426, so that each sensor is in thermal communication with the cooler 482.

With further reference to embodiments of the disclosed invention, additional or alternate temperature sensors (not shown) may be placed adjacent to skin or adjacent to ambient air to further inform temperature regulation, or to directly measure body temperature or ambient temperature, respectively.

While several exemplary embodiments have been described for regulating the temperature of at least one biofluid sensor, it is anticipated that other materials, elements and configurations may also be used, provided the alternative materials, elements or configurations provide chronological assurance and accurate detection and measurement of one or more analytes in a biofluid sample. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

1. A biofluid sensing device configured to be worn on an individual's skin, the biofluid sensing device comprising:
one or more biofluid sensors configured to measure an analyte in a biofluid;
one or more temperature sensors configured to measure a temperature near the one or more biofluid sensors; and
one or more thermal insulators comprising a low thermal conductivity material.

2. The device of claim 1, wherein the one or more thermal insulators form an environment for the biofluid sensor that is thermally isolated.

3. The device of claim 1, further comprising one or more of the following thermal adjustors: a heater; and a cooler.

4. The device of claim 3, wherein the one or more thermal insulators is between the one or more thermal adjustors and the skin.

5. The device of claim 3, wherein the one or more thermal insulators is between the one or more biofluid sensors and ambient air.

6. The device of claim 1, wherein the one or more thermal insulators has one of the following thermal conductivities: <0.5 Watts per meter Kelvin (W/m-K); <0.2 W/m-K; <0.1 W/m-K; and <0.05 W/m-K.

7. The device of claim 1, where said temperature near the one or more biofluid sensors is one of the following temperatures: greater than 37 degrees Celsius (° C.); greater than 39° C.; greater than 41° C.; and greater than 45° C.

8. The device of claim 1, wherein the temperature near the one or more biofluid sensors is within one of the following temperature ranges: ±5° C. of 40° C.; ±1° C. of 40° C.; ±0.2° C. of 40° C.; and ±0.1° C. of 40° C.

9. The device of claim 3, further including one or more thermal conductors between the one or more biofluid sensors and the one or more thermal adjustors.

10. The device of claim 9, wherein the one or more thermal conductors has a thermal conductivity greater than 1 W/m-K.

11. The device of claim 1, further including one or more temperature sensors configured to measure a skin temperature.

12. The device of claim 1, further including one or more temperature sensors configured to measure an ambient air temperature.

13. The device of claim 1, wherein the biofluid is predominantly one of the following: sweat; interstitial fluid; blood; plasma; serum; tears; and saliva.

14. A method of regulating temperature in the biofluid sensing device of claim 1, comprising:
measuring a temperature of a biofluid sensor during device operation;
regulating the temperature of the biofluid sensor to within a selected range of the measured temperature of the biofluid sensor; and
maintaining the temperature of the biofluid sensor within the selected range of the measured temperature of the biofluid sensor for the remainder of device operation.

15. The method of claim 14, where the measured temperature of the biofluid sensor is a calibration temperature at which the biofluid sensor is calibrated.

16. The method of claim 14, wherein the device regulates the temperature of the biofluid sensor to within 20% of the measured temperature of the biofluid sensor.

17. The method of claim 14, where the device maintains the temperature of the biofluid sensor within 0.25° C. of the measured temperature of the biofluid sensor.

18. The method of claim 14, wherein the device maintains the temperature of the biofluid sensor within the selected range of the measured temperature of the biofluid sensor by operating one of the following: a heater; and a cooler.

19. The method of claim 14, further comprising, providing a thermally isolated environment for the biofluid sensor.

20. A biofluid sensing device configured to actively regulate an operating temperature of one or more sensors, the device comprising:
one or more biofluid sensors situated in a thermally isolated environment;
one or more temperature sensors configured to measure a temperature in the thermally isolated environment;
one or more thermal adjustors configured to regulate a temperature in the thermally isolated environment; and
a computer processor.

21. The device of claim 20, wherein the device uses the one or more thermal adjustors to regulate the temperature in the thermally isolated environment in response to a measurement by the one or more temperature sensors.

22. The device of claim 20, wherein the one or more thermal adjustors regulates the temperature in the thermally isolated environment to within a selected range of a calibration temperature of the one or more biofluid sensors.

23. The device of claim 20, wherein the one or more biofluid sensors is one or more of the following: an electrochemical aptamer-based sensor; and an ion-selective electrode sensor.

24. The device of claim 20, wherein the one or more thermal adjustors comprises one or more of the following: a heater; and a cooler.

25. The device of claim 20, wherein the operating temperature is regulated to a calibration temperature for the one or more biofluid sensors.
